(19) United States
(12) Patent Application Publication
(10) Pub. No.: US 2024/0271077 A1
(43) Pub. Date: Aug. 15, 2024

(54) AN AUTOMATED SYSTEM FOR MICROBIAL TESTING

(71) Applicant: Nuha Khalid Alekhmimi, Riyadh (SA)

(72) Inventor: Nuha Khalid Alekhmimi, Riyadh (SA)

(21) Appl. No.: 18/566,738

(22) PCT Filed: Oct. 26, 2021
(86) PCT No.: PCT/IB2021/059856

§ 371 Date: Dec. 4, 2023

(51) Int. Cl. C12M 1/36; B01L 3/00; C12M 1/26; C12M 1/34; G01N 35/00; G01N 35/02

(52) U.S. Cl. CPC C12M 41/48; B01L 3/502715; C12M 33/04; C12M 41/46; G01N 35/0099; G01N 35/028; B01L 2200/025; B01L 2300/0819

(57) ABSTRACT

The invention relates to an automated system for microbial testing, said system comprising: a. a sample processing means to automatically inoculate a test container with a sample to be tested, wherein the test container comprises one or more biosensors and a culture medium for culturing one or more microorganisms in said sample; b. an incubator; c. one or more detection devices to detect output from said one or more biosensors; d. one or more sample transport means to transport said test container to said sample processing means, from said sample processing means to said incubator and from said incubator to said one or more detection devices; and e. a control processor in communication with said sample processing means, said incubator, said one or more detection devices, and said one or more sample transport means to control said microbial testing.

### FIELD OF THE INVENTION

The present invention pertains to the field of microbial testing, in particular an automated system for microbial testing.

### BACKGROUND OF THE INVENTION

Rapid microbial testing is critical for the treatment of infectious diseases. This is not always possible with traditional pathogen detection methods. The advent of new technologies including the use of biosensors for microbial testing as expedited microbial testing. See for example, U.S. Pat. No. 10,436,779 which teaches a biosensor using magnetic particles for pathogen detection. While the use of biosensors may expedite microbial testing, human intervention to prepare the microbial samples for testing may cause delays in the testing. In addition, given the skill required to prepare the microbial samples, this human intervention may introduce errors, including but not limited to incorrect samples, contamination of the sample or poor sample growth.

Accordingly, there is a need in the art for rapid microbial testing while maintaining quality control of the microbial testing process.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an automated system for microbial testing. In accordance with an aspect of the present invention, there is provided an automated system for microbial testing, the system comprising: a. a sample processing means to automatically inoculate a test container with a sample to be tested, wherein the test container comprises one or more biosensors and a culture medium for culturing one or more microorganisms in said sample; an incubator; one or more detection devices to detect output from said one or more biosensors; one or more sample transport means to transport said test container to said sample processing means, from said sample processing means to said incubator and from said incubator to said one or more detection devices; and a control processor in communication with said sample processing means, said incubator, said one or more detection devices, and said one or more sample transport means to control said microbial testing.

### BRIEF DESCRIPTION OF THE FIGURES

These and other features of the invention will become more apparent in the following detailed description in which reference is made to the appended drawings.

FIG. 1 illustrates the automated system for microbial testing of an embodiment of the present invention.

FIG. 2 illustrates a detection device for use in the automated system for microbial testing of an embodiment of the present invention.

FIG. 2 illustrates a detection device for use in the automated system for microbial testing of an embodiment of the present invention.

FIG. 3 illustrates a detection device and test container for use in the automated system for microbial testing of an embodiment of the present invention

FIG. 4 illustrates test containers of an embodiment of the present invention comprising biosensors and a culture medium for culturing one or more microorganisms in said sample

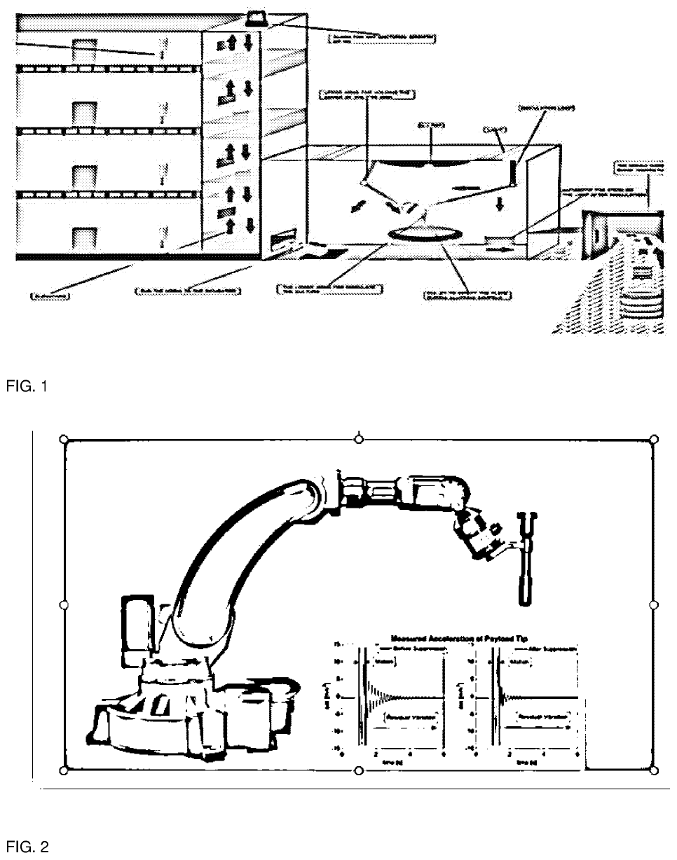

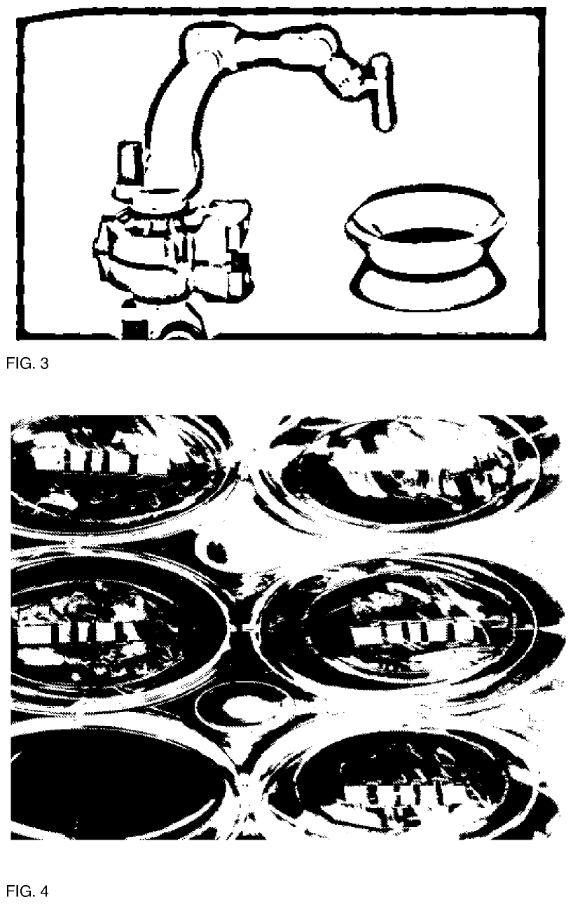

### DETAILED DESCRIPTION OF THE INVENTION

The present invention related to an automated system for microbial testing. The system comprises: a sample processing means to automatically inoculate a test container with a sample to be tested, wherein the test container comprises one or more biosensors and a culture medium for culturing one or more microorganisms in the sample; an incubator; one or more detection devices to detect output from said one or more biosensors; one or more sample transport means to transport said test container to said sample processing means, from said sample processing means to said incubator and from said incubator to said one or more detection devices; and a control processor in communication with said sample processing means, said incubator, said one or more detection devices, and said one or more sample transport means to control said microbial testing. In certain embodiments, the device processes at least 50 samples a day. In certain embodiments, the system allows for high throughput microbial testing.

### Control Processor

The control processor is in communication with the sample processing means, the one or more incubator, the one or more detection devices, and the one or more sample transport means to control the microbial testing. In specific embodiments, the control processor is in wireless communication with the sample processing means, the one or more incubator, the one or more detection devices, and the one or more sample transport means.

The control processor performs the functions by executing instructions stored in a computer readable medium. In certain embodiments, the control processor comprises instructions for microbial testing of specific types of microorganisms. In certain embodiments, the control processor is programmable and allows the microbial testing protocol to be modified.

### Sample Processing Means

The sample processing means comprises one or more sampling tool(s) to transfer the sample(s) to be tested from biological sample(s) to said test container(s). In certain embodiments the sample processing means processes single samples. In other embodiments, the sample processing means allows for batch-processing of samples. The sampling tool may be any sampling tool known in the art for sampling microbial samples. Exemplary sampling tools include but are not limited to an inoculation loop, needle, pipette or swab. In certain embodiments, the sampling tool is an inoculation loop or needle. The sampling tool may be re-usable or disposable. In re-usable embodiments, the sampling tool is sterilized between uses.

In certain embodiments, the one or more sampling tool(s) comprise one or more detection devices. In specific embodiments, the one or more sampling tool(s) comprise a pH sensor. The pH of the sample may be used to determine specimen type and the streaking technique used on the test container.

The one or more sampling tool(s) are attached, optionally removably attached, to one or moveable robotic arm(s). In certain embodiments, the robotic arm(s) comprise one or more means to hold the one or more sampling tool(s). For example, the robotic arm(s) may comprise a grabbing tool to hold the one or more sampling tool(s). The robotic arm is moveable in one or more plans. In certain embodiments, the robotic arm is moveable up and down and can rotate. In certain embodiments, the robotic arm comprises at least one joint, optionally the at least one joints are rotary joints. In specific embodiments, the robotic arm has at least three rotary joints.

The one or more robotic arms are in communication with the control processor. In certain embodiments, the one or more robotic arms are in wireless communication with the control processor. The robotic arm in response to instructions from the control processor and optionally one or more sensors moves to allow the sampling tool to collect said sample to be tested from the biological sample and inoculate said test container. In certain embodiments, the robotic arm is capable of moving to allow one or more different types of inoculations including one or more streaking patterns and/or one or more liquid inoculations. In alternative embodiments, a robotic arm carrying the test container moves the test container to the sampling tool and moves in a pattern to inoculate the test container. In certain embodiments, the robotic arm moves to streak the sample on solid medium in a test container. In certain embodiments, the streaking pattern is based on the data collected by one or more detection devices on the sampling tools. In specific embodiments, the streaking pattern is dependent on the pH of the sample.

In certain embodiments, prior to sample collection, the robotic arm in response to instructions from the control processor grabs a new disposal sampling tool or moves the re-usable sampling tool through a flame of a gas burner for a sufficient period of time to sterilize the sampling tool.

In certain embodiments, when inoculation of the test container is completed, the robotic arm sends a signal to the control processor. If there are no additional samples, the control processor instructs the robotic arm to discard or sterilize the sampling tool and to pause or shut down. If there are additional samples, the control processor instructs the robotic arm to replace or sterilize the sampling tool and initiate collection of a new sample.

In addition, once the test container has been inoculated, the control processor instructs said one or more sample transport means to transport said inoculated test container to said incubator and, optionally instructs said one or more sample transport means to obtain a new test container for inoculation.

### Incubator

The system of the present invention further comprises one or more incubators. The incubators are in communication with the control processor. In certain embodiments, the one or more incubators are in wireless communication with the control processor. In certain embodiments, the incubator comprises sensors at each shelf of the incubator. In certain embodiments, the incubators comprise one or more sensors for real-time monitoring of microbial growth. Exemplary sensors include but are not limited to cameras, humidity sensors, temperature sensors, airflow sensors, CO₂ sensors, photon sensors and optical sensors, fluorescent sensors. In certain embodiments, the incubators are smart devices. In certain embodiments, the incubator comprises one or more photon ray sensors. In certain embodiments, the incubator comprises one or more fluorescent sensors. In certain embodiments, level of microbial growth is determined based on data collected by the one or more sensors.

In certain embodiments, the incubators can modify incubator conditions in response to instructions from the control processor and/or the one or more sensors. In certain embodiments, the humidity of the incubators is maintained at 98%.

In certain embodiments, when a pre-determined level of microbial growth has been detected in the test container inoculated with said sample and/or a pre-determined amount of time has elapsed, the incubator signals to the control processor and the control processor instructs the sample transport means to transport said test container to said one or more detection devices. In specific embodiments, the pre-determined amount of time is 24 hours.

### Sample Transport Means

The automated system further comprises one or more transport means which move one or more test containers through the system in response to instructions from the control processor, signals from one or more sensors and/or one or more other transport means.

The one or more transport means may comprise one or more robotic arms; one or more conveyors and/or one or more elevators/lifts.

### Detection Devices

The automated system further comprises one or more detection devices in communication with the control processor to detect output from the one or more biosensors and/or outputs indicative of microbial growth. The one or more detection devices are configured to detect output automatically or in response to instructions from the control processor. The one or more detection devices comprise one or more of the following: UV resonance raman spectrometer (UVRR), voltage sensor, ultraviolet-visible light spectrophotometer and quadrupole dielectrophoresis (DEP) raman device. In specific embodiments, the one or one or more detection devices comprises a UV resonance raman spectrometer (UVRR), voltage sensor, ultraviolet-visible light spectrophotometer and quadrupole dielectrophoresis (DEP) raman device.

In certain embodiments, the system comprises a single device comprising a photon reader, bacterial cleavage reader, DNA or RNA chip reader and antibiotic reader. In specific embodiments, the device comprises a head housing the readers which can rotate. Bacterial cleavage may be measured using a voltage sensor. Antibiotic reader refers to a quadrupole dielectrophoresis (DEP) Raman device or a infrared detector to measure the zone sensitivity/resistant of the antibiotic discs on the smart-dish plate.

In certain embodiments, the data collected by the one or more detection devices is automatically sent to said control processor, and said control processor sends data to a database and/or generates a report.

### The Test Container

The system is for use with one or more test containers. The test containers comprise one or more biosensors and a culture medium for culturing one or more microorganisms in the sample. In certain embodiments, the test containers are in the form of a petri dish.

In certain embodiments, the culture medium is a solid medium. In specific embodiments, the culture medium is an agar based medium. One or more sugars may be added to enhance microbial growth. In certain embodiments, the culture medium comprises one or more of glucose, fructose, sucrose and maltose. In specific embodiments, the culture medium comprises glucose, fructose, sucrose and maltose. In certain embodiments, the culture medium comprises one or more components to facilitate visualization of bacterial colonies. For example, a brilliant green indicator facilitates the visualization and counting of gram-positive bacteria colonies and neutral red may be used for differentiating between gram negative bacteria lactose fermenter and non-lactose fermenter.

In specific embodiments, the culture medium comprises per litre:

| Ingredients | Gram/liter | |
|---|---|---|
| Peptone (Pancreatic digest of gelatin) | 10.0 | gm |
| Tryptose | 10.0 | gm |
| Sodium chloride | 5.0 | gm |
| Lactose monohydrate | 10 | gm |
| Crystal Violet | 0.001 | g |
| Neutral red | 0.03 | gm |
| Glucose, fructose, sucrose and maltose | 10 g for each | |
| Brilliant green | 7 | mg |
| Agar | 15 | gm |

| | |
|---|---|
| Agarose | 1% for long shelf life |
| Distilled Water | Add to make 1 Liter |

Final pH 7.1+/−0.2 at 25 degrees C.

The test container comprises one or more biosensors. one or more biosensors are a nucleic acid microarray, protease sensor and microbial colorimetric bead assay.

In certain embodiments, the test container comprises a nucleic acid microarray specific for one or more microorganisms including but not limited to particular gram positive and/or gram-negative bacteria. Appropriate probes for use in the nucleic acid microarray can be readily determined by a worker skilled in the art. For example, probes may be designed based on publicly available databases such as Genbank. In certain embodiments, the probes may have high-specificity or may target conserved regions. The nucleic acid microarray may be a DNA microarray or a RNA microarray. In specific embodiments, the nucleic acid microarray is a paper-based microarray. In specific embodiments, the nucleic acid microarray is a chip-based microarray. In certain embodiment, the test container comprises an electrochemical biosensor (electrochemical impedance spectroscopy) using voltage sensor.

In certain embodiments, the test container comprises a paper-based protease sensor. As used herein protease sensor refers to a sensor for detecting protease activity Exemplary proteases include but are not limited to MMP2, PR3, NE, 20 LL37, α- Defensin, MMP9 and intgron 1/integron 2, integrins, MPO, Histons, entraxin, lactoferrin, gelatinase, cathepsin G. In specific embodiments, the biosensor Aluminum foil/Silver surface was functionalized with a layer of the black color protease substrate- magnetic nanoparticles (MNPs) composite which upon the protease's cleavage the black color turn to silver color. A non-limiting method of producing the protease sensor is at follows: Initially, the substrate-magnetic nanoparticles (MNPs) composite suspension was mounted over the Aluminum foil/Silver sensor surface and allowed to stand at room temperature for 30 min. Subsequently, an external magnet (10×10×5 mm) with a field strength of 3300 gauss at 2 mm distance, respectively, was passed over the functionalized strip to remove any unattached substrate-MNPs conjugates. At this stage, the sensor surface silver color is masked and turned black. After that, round paper magnet was fixed on the back of the strip, 2-3 mm distance below the sensor platform.

In certain embodiments, the test container comprises colorimetric beads specific for a particular group of pathogens. In certain embodiments, colorimetric beads target gram-positive bacteria. In certain embodiments, the colorimetric beads target gram-negative bacteria. In certain embodiments, the test container comprises colorimetric beads targeting gram-positive bacteria and other colorimetric beads (a different colour) target gram-negative bacteria. In specific embodiments, aptamers to a particular target sequence are conjugated to coloured polymer beads. For example, an aptamer targeting LPS may be conjugated to a blue polymer nano bead to target gram negative bacteria. A non-limiting method of conjugating the aptamer to nanobeads and mixed with agar is detailed below: 300 nm spheres, colored polymer (nano) beads was washed with distilled water several times before being reacted with a coupling mixer of EDC/NHS over night; then, the beads were washed in distilled water to remove excess coupling agent. LPS aptamer was linked to activated blue polymer beads. Blocking of any unbound sites was achieved using 1% BSA solution in PBS for 30 min. Finally, Agar of bacterial media mixed colored polymer (nano) beads at room temperature. A different colour bead such as orange 200 nm spheres are used for gram positive bacteria Aptamer). Unbound beads are removed by incubating the activated beads into a well-mixed solution of antibody or aptamer with Phosphate buffered saline (PBS) and incubated overnight at 4° C. The Conjugation beads was washed to removed away any unbounded antibody and thereafter Bovine Serum Albumin1% (BSA) was added to block the active sites on the beads. Peptides coupling buffer was immobilized onto beads for 30 min at room temperature.

In specific embodiments, the test container comprises a nucleic acid microarray, protease sensor and microbial colorimetric bead assay.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An automated system for microbial testing, said system comprising:
a. a sample processing means to automatically inoculate a test container with a sample to be tested, wherein the test container comprises one or more biosensors and a culture medium for culturing one or more microorganisms in said sample;
b. an incubator;
c. one or more detection devices to detect output from said one or more biosensors;
d. one or more sample transport means to transport said test container to said sample processing means, from said sample processing means to said incubator and from said incubator to said one or more detection devices; and
e. a control processor in communication with said sample processing means, said incubator, said one or more detection devices, and said one or more sample transport means to control said microbial testing.

2. The automated system of claim 1, wherein said sample processing means comprises a sampling tool to transfer said sample to be tested from a biological sample to said test container.

3. The automated system of claim 2, wherein said sampling tool is an inoculation loop or needle.

4. The automated system of claim 3, wherein said inoculation loop or needle is attached to a moveable robotic arm; wherein said robotic arm is in communication with said control processor and wherein in response to instructions from said control processor said robotic arm moves to allow said sampling tool to collect said sample to be tested from said biological sample and inoculate said test container.

5. The automated system of claim 3, wherein said inoculation loop or needle comprises one or more of said detection devices.

6. The automated system of claim 5, where said inoculation loop or needle comprises a pH sensor.

7. The automated system of claim 4, wherein said robotic arm signals to said control processor when inoculation of said test container is completed and said control processor instructs said robotic arm to replace said inoculation loop or needle with a new inoculation loop or needle; and said control processor instructs said one or more sample transport means to transport said inoculated test container to said incubator and, optionally instructs said one or more sample transport means to obtain a new test container for inoculation.

8. The automated system of claim 1, wherein said one or more transport means comprises one or more robotic arms; one or more conveyors and/or one or more elevators to move said test container in response to instructions from said control processor.

9. The automated system of claim 1, wherein incubator comprises one or more sensors for real-time monitoring of microbial growth.

10. The automated system of claim 9, wherein said incubator signals to said control processor when a pre-determined level of microbial growth has been detected in said test container inoculated with said sample and/or a pre-determined amount of time has elapsed and said control processor instructs said sample transport means to transport said test container to said one or more detection devices.

11. The automated system of claim 1, wherein said one or more detection devices comprises one or more of the following: UV resonance raman spectrometer (UVRR), voltage sensor, ultraviolet-visible light spectrophotometer and quadrupole dielectrophoresis (DEP) raman device.

12. The automated system of claim 11, wherein said one or more detection devices comprises a UV resonance raman spectrometer (UVRR), voltage sensor, ultraviolet-visible light spectrophotometer and quadrupole dielectrophoresis (DEP) raman device.

13. The automated system of claim 1, wherein data collected by said one or more detection devices is automatically sent to said control processor, and said control processor sends data to a database and/or generates a report.

14. The automated system of claim 1, wherein said one or more biosensors are a nucleic acid microarray, protease sensor and microbial colorimetric bead assay.

15. The automated system of claim 14, wherein said nucleic acid microarray is a paper-based DNA microarray or a chip-based DNA microarrary.

16. The automated system of claim 1, wherein said culture medium comprises per litre:

| Ingredients | Gram/liter | |
|---|---|---|
| Peptone (Pancreatic digest of gelatin) | 10.0 | gm |
| Tryptose | 10.0 | gm |
| Sodium chloride | 5.0 | gm |
| Lactose monohydrate | 10 | gm |
| Crystal Violet | 0.001 | g |
| Neutral red | 0.03 | gm |
| Glucose, fructose, sucrose and maltose | 10 g for each | |
| Brilliant green | 7 | mg |
| Agar | 15 | gm |
| | |
|---|---|
| Agarose | 1% for long shelf life |
| Distilled Water | Add to make 1 Liter |
